(19) United States
(12) Patent Application Publication
(10) Pub. No.: US 2008/0243095 A1
(43) Pub. Date: Oct. 2, 2008

(54) Method and Apparatus for Ophthalmic Medication Delivery and Ocular Wound Recovery

(75) Inventors: Richard S. Kaiser, Plymouth Meeting, PA (US); Jonathan L. Prenner, Princeton, NJ (US)

Correspondence Address: PEPPER HAMILTON LLP, ONE MELLON CENTER, 50TH FLOOR, 500 GRANT STREET, PITTSBURGH, PA, 15219, US

(73) Assignee: THETA RESEARCH CONSULTANTS, LLC, Plymouth Meeting, PA (US)

(21) Appl. No.: 12/055,644

(22) Filed: Mar. 26, 2008

Related U.S. Application Data
(60) Provisional application No. 60/982,965, filed on Oct. 26, 2007.
(60) Provisional application No. 60/896,998, filed on Mar. 26, 2007.
(60) Provisional application No. 60/954,730, filed on Aug. 8, 2007.

(51) Int. Cl. A61F 9/00; A61M 35/00

(52) U.S. Cl. 604/294

(57) ABSTRACT

An annular device may include a biocompatible material. The device may be configured to substantially conform to a curvature of an eye. The device may be placed on a surface of the eye such that the device surrounds, but does not cover, the cornea. The device may cover a site of one or more incisions in a sclera of the eye without extending past an edge of a bulbar conjunctiva of the eye.

### RELATED APPLICATIONS AND CLAIM OF PRIORITY

This patent application claims priority to U.S. Provisional Patent No. 60/896,998, entitled Ophthalmic Antibiotic Delivery Device, filed on Mar. 26, 2007; U.S. Provisional Patent No. 60/982,965, entitled Method and Apparatus for Ophthalmic Medication Delivery and Ocular Wound Recovery, filed Oct. 26, 2007; and U.S. Provisional Patent No. 60/954,730, entitled Ophthalmic Medication Delivery Device, filed on Aug. 8, 2007.

Not Applicable

### BACKGROUND

Various surgical procedures are routinely performed on the human eye. For example, cataract surgery involves making an incision in the cornea and removing the lens. Another ocular surgical procedure is a pars plana vitrectomy which involves making multiple small incisions in the conjunctiva and sclera of the eye to remove the vitreous humor and manipulate the retina and epi-retinal tissue in the posterior segment of the eye. The vitrectomy incisions are made through the pars plana, and generally made about 3.5 millimeters outside the outer edge of the cornea, which is itself about 12.5 millimeters in diameter in the adult human eye.

Vitrectomy surgery has undergone recent technological advances that have enabled surgeons to perform trans-conjunctival and trans-scleral incisions with 23-gauge or 25-gauge blades, which measure just millimeters in diameter. These new techniques (23-gauge and 25-gauge vitrectomy) are more sophisticated and are gaining popularity among vitreo-retinal ophthalmic surgeons. Micro-incision surgery is slowly replacing 20-gauge surgery, a vitrectomy approach that utilizes larger operative incisions that are sutured at the end of surgery. There are multiple advantages of the micro-incision techniques (23-gauge and 25-gauge) including that the wounds can be self-sealing and typically do not require sutures at the end of the surgical procedure. As such, post-operative patient comfort is improved and operation and recovery times are reduced.

Endophthalmitis is a complication that may result from ophthalmic procedures, including vitrectomy procedures. Endophthalmitis is a pan-ophthalmic infection of the eye that frequently results in blindness or severe loss of vision and, on occasion, can require removal of the eye. Despite the advantages of micro-incision eye surgery, such as vitrectomy, such procedures still result in an unfortunate number of incidents of post-operative endophthalmitis in patients. Although endophthalmitis is more prevalent following micro-incision surgery than following other ocular surgical procedures, ophthalmic surgeons typically perform micro-incision surgeries because of the numerous advantages it provides to patients. Therefore, it is desirable to prevent and control endophthalmitis following micro-incision surgical procedures, such as vitrectomy.

One way to reduce the incidence complications from ocular surgical procedures is to insert a drug delivery device post-operatively. Some present devices for post-operative drug delivery designed for cataract surgery consist of small disc-shaped shields designed to cover centrally-located corneal incisions. However, such devices are not substantially useful for use following a vitrectomy because the devices would not cover surgical wounds in the sclera.

### SUMMARY

In an embodiment, a method for administering ophthalmic medication to a site of one or more incisions in a sclera of an eye may include applying an annular device to the site. The device may include a biocompatible material, an inner edge and an outer edge. The inner edge may surround a cornea of the eye without covering the cornea and the outer edge may cover the site of the one or more incisions without extending past an edge of a bulbar conjunctiva of the eye. The device may provide medication to at least the site of the one or more incisions.

In an embodiment, an annular device may include a biocompatible material and may be configured to substantially conform to a curvature of an eye. The device may be configured to be placed on a surface of the eye such that the device surrounds, but does not cover, the cornea. The device may also cover a site of one or more incisions in a sclera of the eye without extending past an edge of a bulbar conjunctiva of the eye.

In an embodiment, an annular device may include a biocompatible material and an ophthalmic medication. The device may have an inner edge and an outer edge and may be configured to substantially conform to a curvature of an eye. The device may be configured to be placed on a surface of the eye such that the inner edge surrounds, but does not cover, the cornea and the outer edge covers a site of one or more incisions in a sclera of the eye without extending past an edge of the a bulbar conjunctiva of the eye. The device may be further configured to release the ophthalmic medication over a period of time to at least the site of the one or more incisions when the device is placed on the surface of the eye.

In an embodiment, an annular device may include a biocompatible material and an absorbed amount of an ophthalmic medication. The device may have an inner edge having a diameter of at least about 10 millimeters and an outer edge having a diameter of no more than about 26 millimeters. The device may be configured to substantially conform to a curvature of an eye. The device may be configured to be placed on a surface of the eye such that the inner edge surrounds, but does not cover, the cornea and the outer edge covers a site of one or more incisions in a sclera of the eye without extending past an edge of a bulbar conjunctiva of the eye. The device may release the ophthalmic medication over a period of time to at least the site of the one or more incisions when the device is placed on the surface of the eye.

In an embodiment, an annular device may include a biocompatible material and an absorbed amount of an ophthalmic medication. The device is configured to substantially conform to a curvature of an eye. The device may have an inner edge and an outer edge and may be configured to be placed on a surface of the eye such that the inner edge surrounds, but does not cover, the cornea. The inner edge may define a substantially circular shaped central opening, and the outer edge may cover a site of one or more incisions in a sclera of the eye without extending past an edge of a bulbar conjunctiva of the eye. The outer edge may have an irregular shape. The device may release the ophthalmic medication over a period of time to at least the site of the one or more incisions when the device is placed on the surface of the eye.

### BRIEF DESCRIPTION OF THE DRAWINGS

Aspects, features, benefits and advantages of the embodiments described herein will be apparent with regard to the following description, appended claims, and accompanying drawings where:

FIG. 1 illustrates an exemplary sagittal cross section of a human eye.

FIG. 2 illustrates an exemplary view of a human eye.

FIG. 3 illustrates an exemplary frontal view of a treatment device according to an embodiment.

FIG. 4 depicts a frontal view of an eye having an exemplary device placed therein according to an embodiment.

FIG. 5 illustrates an exemplary embodiment of an annular device where one of the edge of the device is non-circular according to an embodiment.

FIG. 6 illustrates an exemplary embodiment of a star-shaped device according to an embodiment.

FIG. 7 illustrates an exemplary embodiment of a U-shaped device according to an embodiment.

FIG. 8 depicts a side view of an exemplary device as placed on the surface of a human eye according to an embodiment.

### DETAILED DESCRIPTION

An ophthalmic device may be used to reduce the incidence of, for example, endophthalmitis. In particular, the device may be used following micro-incision vitrectomies (23-gauge and 25-gauge) and may be used to reduce endophthalmitis and other reported complications associated with vitrectomy procedures.

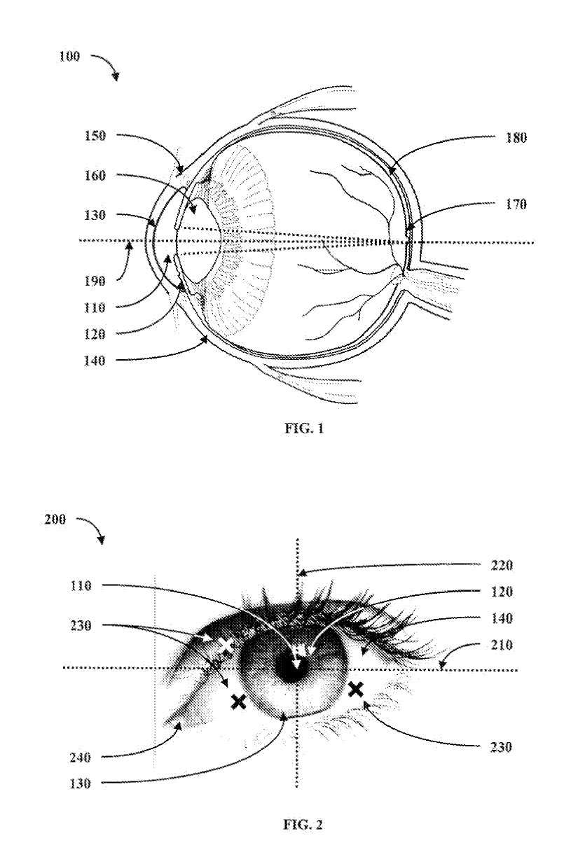

FIG. 1 illustrates an exemplary sagittal cross section of a human eye. Some major parts of the eye 100 are indicated, including the pupil 110, iris 120, cornea 130, sclera 140, conjunctiva 150, lens 160, macula 170 and retina 180. The approximate central vision axial line of sight 190 is also depicted in FIG. 1.

FIG. 2 illustrates an exemplary view of a human eye. The eye 200 is shown as it appears when looking towards a subject. The center of the pupil 110 can be considered the central part of the eye 200, from which radial measurements can be made. As such, two exemplary orthogonal axes of symmetry 210 and 220 can be drawn. Axis 210 corresponds to a transverse plane with respect to the human subject, while axis 220 corresponds to a sagittal plane with respect to the human subject.

Considering the pupil 110 to be at or near the center of the eye 200 and the eye to have a substantially radially symmetric geometry, the cornea 130 extends to about 12 millimeters from the center of tile eye. It is to be appreciated that an eye 200 is not flat or absolutely circular; rather, an eye possesses a curvature away from a two-dimensional plane. Additionally, it is to be appreciated that the cornea 130 has a higher degree of curvature than the sclera 140. For example, the cornea 130 may have a base radius of curvature of about 8 millimeters, while the sclera 140 may have a base radius of curvature of about 12.25 millimeters. However, for the present purposes, the dimensions of the eye 200 and the device may be measured substantially in the plane of the drawing with the center of the idealized eye being at about the origin of the radial symmetry of the eye.

An ophthalmic device may be adapted to the conjunctival and scleral regions, posterior to the corneal limbus. For example, the device may be used to cover micro-incision sites left by vitrectomy procedures, indicated (not to scale) by cross marks 230 on FIG. 2. Incisions 230 are typically located approximately 3.5 millimeters outside the periphery of the cornea. FIG. 2 further depicts the caruncle 240 for orientation purposes.

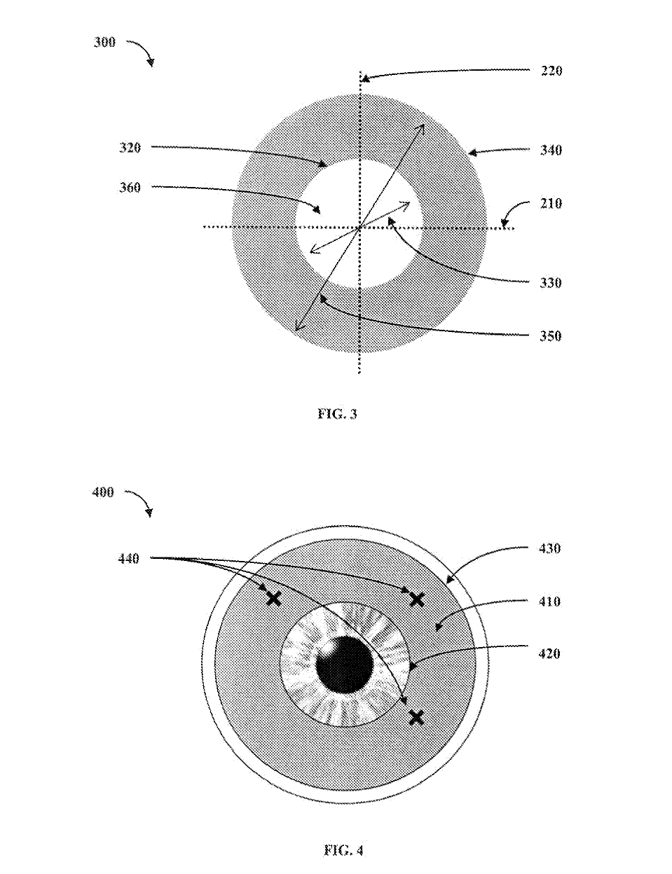

FIG. 3 illustrates an exemplary frontal view of a treatment device 300 according to an embodiment. As shown in FIG. 3, the device 300 is an annular device. In an embodiment, the device 300 may be formed of a biocompatible material. The biocompatible material may include, but is not limited to, collagen, poly(methylmethacrylate), poly(propylene), poly(ethylene), silicone, poly(tetrafluoroethene), fluoropolymers, poly(etheretherketone) and/or nylon. In an embodiment, the biocompatible material may include hydrogels such as, but not limited to, carboxymethyl cellulose, poly(vinyl alcohol), carboxymethyl starch, poly(acrylic acid), poly(saccharides) and/or poly(lactic acid) copolymers.

In an embodiment in which the biocompatible material comprises collagen, the biocompatible material may comprise crosslinked collagen and partially hydrolyzed collagen. In an embodiment, the device 300 may comprise high purity, low immunogenic, type I collagen. In an embodiment, the device 300 may comprise at least 95% high purity type I collagen. In an embodiment, the device 300 may comprise at least 98% high purity type I collagen. In an embodiment, the device 300 may comprise a hydrogel construct.

As shown in FIG. 3, the device 300 may be generally rounded in shape with a center configured to be placed approximately at the intersection of axes of symmetry 210 and 220 of an eye 200, as discussed above in reference to FIG. 2. The device 300 may be generally annular in form. However, the device 300 is not limited to rounded or circular embodiments, even though these may be preferred in some instances. In an embodiment, the device 300 may be configured to be placed on the surface of the eye such that the device surrounds, but does not cover, the cornea.

The device 300 may include an inner edge 320 defined by an inner diameter 330 and an outer edge 340 defined by an outer diameter 350. An outer diameter 350 may generally have a greater length than the inner diameter 330. As mentioned elsewhere, the actual profiles and shapes of various embodiments, as well as the sizing of the various dimensions of the various embodiments will vary depending on a number of factors. For example, the type of surgical procedure, the size of the eye, the location of the incisions, and other design and clinical considerations may be taken into account when designing a device.

In one or more embodiments, the inner edge 320 and the outer edge 340 may each be substantially circular and may have a common center point (at the intersection of axes 210, 220). The center point may be located on or around the pupil when the device 300 is placed on an eye. In an embodiment, the inner edge 320 and outer edge 340 of the annular device 300 may be concentric or approximately concentric.

In an embodiment, the device 300 may have an opening or aperture 360. Note that an annulus is typically recognized as a region between two concentric circles, but the present annular embodiments are intended to be more generalized to cover, for example, device designs that are not necessarily circular in shape, and embodiments where the inner and outer edges 320 and 340 arc not strictly concentric, although concentric circular designs may be preferred in some applications. This flexibility in shape and size of the device may include many possible geometries and dimensions (or diameters) for the inner 320 and outer edges 340 of the device 300.

In an embodiment, the device may include an inner edge 320 defining a substantially circular shaped central opening having an inner diameter 330 between about 0.1 millimeters (mm) and about 10 mm. In an embodiment, the inner diameter 330 may be between about 10 mm and about 14 mm. In an embodiment, the inner diameter 330 may have a diameter greater than about 14 mm. In an embodiment, the inner diameter 330 may cover a portion of the cornea if, for example, such coverage is anticipated to provide added or beneficial protection or drug delivery to the eye.

In an embodiment, the device 300 may further include an outer edge 340. In an embodiment, the outer diameter 350 may be configured to cover micro-incisions in the sclera resulting from ocular surgery. In an embodiment, the outer diameter 350 of the device 300 may cover the site of an incision in the sclera without extending past the edge of the bulbar conjunctiva of the eye. In an embodiment, the outer diameter 350 of the device may have a diameter dimensioned to cover up to and including a diameter of about 26 mm. More particularly, the device 300 may have an outer diameter 350 between about 14 mm and about 26 mm. In a preferred embodiment, the outer diameter 350 may be between about 24 mm and about 25 mm.

In an embodiment, a distance between the inner edge 320 and the outer edge 340 may be between about 2 mm and about 8 mm. In an embodiment, a distance between the inner edge 320 and the outer edge 340 may be about 6 mm. In an embodiment, a distance between the inner edge 320 and the outer edge 340 may be about 10 mm.

FIG. 4 depicts a frontal view of an eye having an exemplary device placed therein according to an embodiment. An exemplary device 410 is shown as applied to the eye 400 for post-operative treatment of surgical incisions 440. Incisions 440 are disposed in the sclera of the eye at a radial distance outside the cornea and iris of the eye. Specifically, the illustrative example shows the incisions 440 at a radial distance of 3.5 mm outside the cornea, where such incisions would be unprotected and untreated with previously-known devices and prone to endophthalmitis or similar maladies absent the teachings disclosed herein. In FIG. 4, the device, defined by the inner edge 420 and outer edge 430, may cover the incisions 440. The device 410 may be configured to substantially conform to a sclera base radius of curvature.

In an embodiment, the device 410 may include an absorbent material, such as collagen, and have a substantially circular outer edge 430 that is capable of covering vitrectomy surgical incisions, a site of an intravitreal injection, or a site of an implantation posterior to the corneal edge of the eye. Optionally, the absorbent material of the device 410 may absorb an ophthalmic medication. In an embodiment, the medication may be an antibiotic solution or suspension that may be slowly released over, for example, the first twenty-four hours after surgery. Optionally, the device 410 may dissolve over a variable length of time. In an embodiment, the device 410 may dissolve on its own when in contact with surface eye fluids (tears) over a period of about twelve hours to about forty eight hours after placement in the eye (i.e., after surgery). In an embodiment, a ratio of crosslinked collagen to partially hydrolyzed collagen may determine the dissolving period. In some embodiments, the dissolving process may obviate the need for a follow-up procedure to remove the device 410. Alternatively, the device 410 may not dissolve and may need to be removed.

Optionally, the device 410 may include an absorbed amount of ophthalmic medication. In an embodiment, the ophthalmic medicine may include, but is not limited to, an antibiotic, a chemical, a vitamin, a balm and/or another substance or substances absorbed into the device. In an embodiment, the ophthalmic medication is an antibiotic solution or suspension which may be applied on the device 410 or ophthalmic shield prior to placement of the device on the surface of the eye. In an embodiment, the ophthalmic medication is an antibiotic solution or suspension which may be applied on the device 410 or ophthalmic shield after placement of the device on the surface of the eye. Thus, one or more embodiments of the ophthalmic medication delivery device 410 include an ophthalmic antibiotic. Non-limiting examples of suitable antibiotics include penicillins, cephalosporins, tobramycin, ciprofloxacin, ofloxacin, levofloxacin, moxifloxacin, gatifloxacin and fourth generation fluoroquinolones. It will be appreciated by one of skill in the art that the choice of antibiotic or antibiotics used is not critical and is merely according to the surgeon's preference.

Non-limiting examples of medications suitable for delivery by the ophthalmic medication delivery device 410 include not just antibiotics, but also antimicrobials, antivirals, antifungals, antiparasitics, steroids, non-steroidal medications and/or other ocular medications including medications for treating retinal conditions, such as age-related macular degeneration, diabetic retinopathy, vascular occlusions of the retina, retinal degenerative diseases (including but not limited to retinitis pigmentosa), retinopathy of prematurity and other retinal diseases.

The ophthalmic medicine may be controllably released into areas of the eye at or near the eye's surface. Therefore, embodiments may include variations in dosage, concentration, composition and/or other characteristics of the medication to be applied to the eye. This can be reflected in the way the medication is introduced into the device 410 (e.g., its local concentration and by the amount of time the medication is applied to the eye.

Optionally, the medication may be released over a variable length of time depending on the medication and/or the material of the device. In an embodiment, the device 410 may allow medication to be slowly released by a collagen material for a constant release onto the surface of the conjunctiva and sclera over a period of a few hours to a few days depending on the type of collagen or other material used. In an embodiment, the device may be configured to limit the release of the medication to locations posterior to the cornea. In an embodiment, other materials besides collagen may be useful for delivery of medications to the surface of the eye posterior to the cornea.

Optionally, the device 410 may release the ophthalmic medication over a period of time to at least the site of the incision when the device is placed on the surface of the eye. In an embodiment, the device 410 may be designed to time-release the absorbed medication. For example, complete delivery of the medication may take from about twenty-four hours to about seventy-two hours to provide effective treatment to the eye. In an embodiment, complete delivery of the medication may take from about twenty-four hours to about forty-eight hours. In an embodiment, complete delivery of the medication may take from about twenty-four hours to about thirty-six hours. In an embodiment, complete delivery of the medication may take less than about twenty-four hours.

In an embodiment, the device 410 may be made of a soluble material, such as a collagen, that can gradually dissolve or be absorbed or washed away by the body's fluids and in the subject's eye. In an embodiment, the device 410 may be dissolvable. In an embodiment, the device 410 may completely erode within about twenty-four hours to about seventy-two hours. In an embodiment, the device 410 may completely erode within about twenty-four hours to about forty-eight hours. In an embodiment, the device 410 may completely erode within about twenty-four hours to about thirty-six hours. In an embodiment, the device 410 may completely erode within about twenty-four hours.

The utility of the ophthalmic medication delivery device 410 extends beyond its use following suture-less vitrectomy surgery. The use of the present delivery device 410 includes, but is not limited to, use after intravitreal injection of any mediation or material, use following the implantation of a material or a device through the conjunctiva and/or the sclera, and use following a procedure performed in ophthalmology or optometry that benefits from the delivery of an antibiotic or medication. Some specific embodiments provide the antibiotic or medication posterior to the corneal limbus of the eye.

In an embodiment, the device 410 may include a disinfectant or active ingredient such as, but not limited to, an anesthetic. An anesthetic may include, but is not limited to, lidocaine, tetracaine (amethocaine), prilocaine, benzocaine, bupivacaine, cocaine, etidocaine, mepivacaine, pramoxine, prilocaine, procaine, proparacaine, ropivacaine and mixtures thereof. In an embodiment, an anesthetic may be used prior to an ocular procedure or surgery. Optionally, the device 410 may include biologically active agents, such as, but not limited to antimicrobial fungicides or virocides. In an embodiment, the device 410 may include antibiotics, such as, but not limited to, amoxicillin, ampicillin, cefaclor, clarithromycin, ceftriaxone, cefprozil, gentamicin sulfate and/or vancomycin.

In an embodiment, the device 410 may be transparent and/or translucent. In an embodiment, the device is transparent and translucent such that the site of the incision in the sclera of the eye may be viewed through the device.

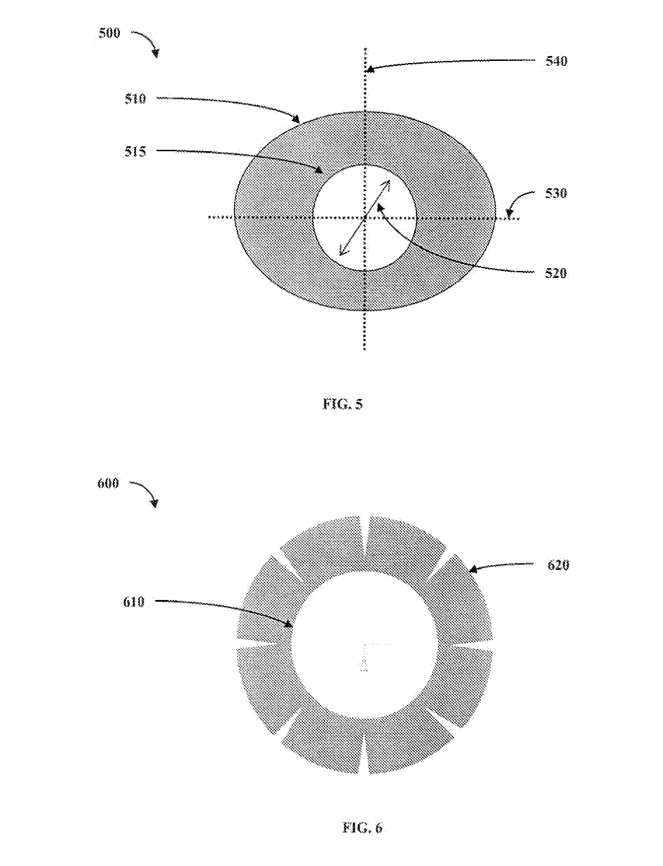

FIGS. 5-7 depict exemplary alternative shapes of the device according to various embodiments. FIG. 5 illustrates an exemplary embodiment of a device 500 having an inner edge 515 with a generally circular profile having a diameter 520 and an outer edge 510 that is substantially ellipsoidal, ovular, or otherwise non-circular. In other words, with reference to the FIG. 5, the inner edge 515 of the device 500 may have approximately the same dimensions along axes 530 and 540, but the outer edge 510 may have different dimensions along axes 530 and 540. In an embodiment, the inner 515 and outer edges 510 may be non-concentric. It can be appreciated that a large number of such embodiments are possible, and may be used in certain contexts as called for by the patient's condition or as otherwise required.

In an embodiment, the device may have a caruncular cutout. For example, the device may be removed so the device does not come into contact with the caruncle of the eye.

In an embodiment, a device may be designed such that the outer edge of the device is not circular. For example, FIG. 6 discloses an exemplary star-shaped device according to an embodiment. As with the exemplary devices disclosed above, the device 600 may be made of a biocompatible material and may have an absorbed amount of an ophthalmic medication. Similarly, the device 600 may substantially conform to a curvature of an eye.

In an embodiment, the star-shaped device may include an inner edge 610 and an outer edge 620. In an embodiment, the inner edge 610 may surround, but not cover, the cornea. The inner edge 610 may define a substantially circular shaped central opening. The inner edge 610 of the device 600 may have a diameter between about 0.1 mm and about 14 mm. In a preferred embodiment, the inner edge 610 of the device 600 may have a diameter between about 10 mm and about 14 mm.

In an embodiment, the outer edge 620 may be designed to cover an incision in a sclera without extending past the edge of the bulbar conjunctiva of the eye. In an embodiment, the outer edge 620 may have an irregular shape. As shown in FIG. 6, the irregular shape may be substantially star-shaped. In an embodiment, the device 600 is star-shaped when two-dimensional, but at least one portion of the star may contact an adjacent portion when the device is fitted to the eye to create a continuous outer edge. In an embodiment, ophthalmic medication may be released over a period of time to the site of the incision when the device 600 is placed on the surface of the eye.

In an embodiment, an annular device 600 may include a biocompatible material and an absorbed amount of an ophthalmic medication. The device 600 may have an inner edge 610 and an outer edge 620. The device 600 may be configured to substantially conform to a curvature of an eye. The device 600 may be placed on a surface of the eye such that the inner edge 610 surrounds a cornea of the eye without covering the cornea. The inner edge 610 may define a substantially circular shaped central opening. The outer edge 620 may cover a site of one or more incisions in a sclera of the eye without extending past an edge of a bulbar conjunctiva of the eye. The outer edge 620 may have an irregular shape. The device 600 may be configured to release the ophthalmic medication over a period of time to at least the site of the one or more incisions when the device 600 is placed on the surface of the eye.

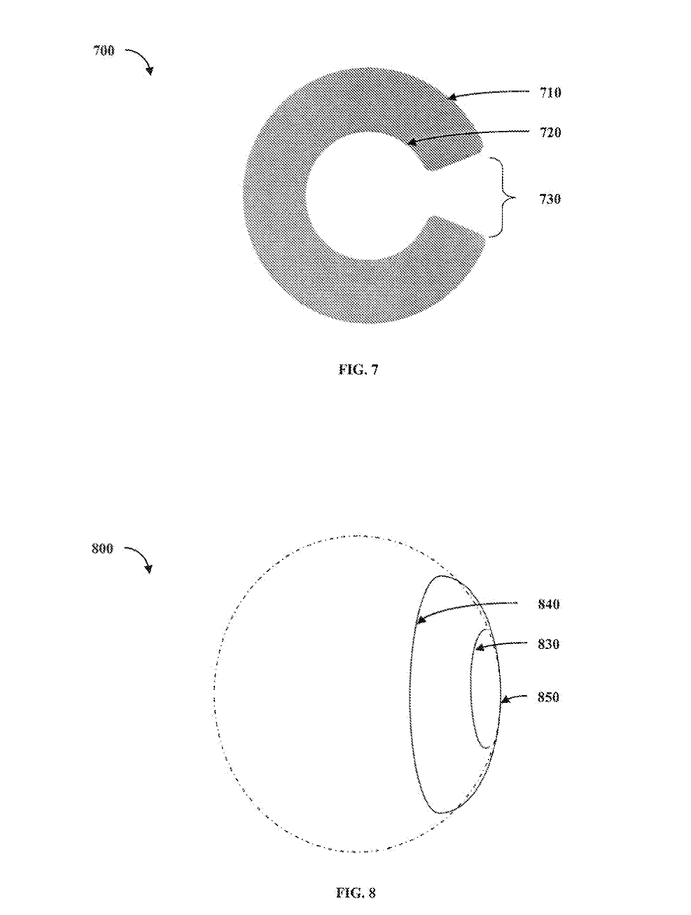

FIG. 7 discloses an exemplary device with an open-ended ring-like structure. In an embodiment, the device 700 may be U-shaped. In an embodiment, the device 700 may include an inner edge 710 and an outer edge 720. In an embodiment, the device 700 may have an open-ended ring-like structure having a cut-out 730 configured to be placed around the caruncular. The open-ended ring-like structure may be molded to and comfortably sit on a patient's eye.

FIG. 8 depicts a side view of an exemplary device placed on the surface of a human eye. The device 800 may be shaped to generally follow the curvature of the eye to improve the fit of the device when placed on the surface of the eye and to minimize wrinkles, buckling, folds, creases, or other artifacts that could result from applying a flat annular shield to a non-flat eye surface. Those skilled in the art will recognize that a metric for measuring eye curvature exists, and such or similar metrics can be used to describe and assist the design of some dimensions of the present device. FIG. 8 depicts how the inner edge 830 and the outer edge 840 of the annulus 800 would generally fit over the eye, and how, in the annular embodiments hereof, some central portion 850 (e.g., the pupil or the cornea) may remain exposed and not covered by the absorbent collagen shield 800.

The devices generally described herein may include any number of design features which add to the mechanical integrity of the device, improve comfort for the patient on whom the device is deployed, improve drug delivery and/or the like. For example, in one embodiment, the inner edge 830 may be reinforced with, for example, a rim material from the device that is more densely packed or a rim of a secondary material having density greater than the material of the device. Alternatively or additionally, the inner edge 830 may include a thickened ridge or geometry. Without wishing to be bound by theory, such reinforcements may provide mechanical resistance to deformation during, for example, blinking. In another embodiment, the inner edge 830 and/or the outer edge 840 may be shaped such that the edge is tapered, beveled, filleted, curved or molded or machined to take on any shape desired. Such shaped edges may provide a dynamic transition zone that lowers the mechanical resistance profile of the device and/or reduces the incidence of the device becoming dislodged. Shaped edges may also provide improved comfort to the patient by reducing resistance to blinking and/or reducing the sensation of a foreign body being in the eye. In an embodiment, the device may include an astute tapered angle between about 0 degrees and about 90 degrees from the outer edge to the inner edge along a central axial length corresponding to a line of sight of the eye. In an embodiment, the device may be tapered to closely fit on the eye. The tapered device may include at least one portion having a straight line configuration. In an embodiment, the device may be tapered to closely fit on the sclera.

In an embodiment, the device may conform to the contour of the eye. In an embodiment, the device may include a sclera base radius of curvature that conforms to the contours of the sclera. In an embodiment, the device may conform to the contour of the sclera. In an embodiment, the device may include a sclera base radius of curvature less than about 13 mm. In a preferred embodiment, the device may include a sclera base radius of curvature between about 9 mm and about 13 mm. In an embodiment, the base radius of curvature may depend on the length of the inner diameter. For example, in an embodiment, the device may include a sclera base radius of curvature between about 9 mm and about 13 mm if the inner diameter is at least about 10 mm.

In an embodiment, the thickness of the device may be substantially uniform. For example, the thickness of the device may be substantially uniform from the inner edge to the outer edge. In an embodiment, the thickness of the device may vary such that the profile of the device is non-uniform. Without wishing to be bound by theory, varying the thickness of the device may provide greater mechanical stability or resistance to deformation and/or may provide reduced resistance to blinking, reduced foreign body sensation and reduced incidence of the device becoming dislodged. Varying the thickness of the device may also be used to effect the active agent dosage and/or the delivery of the device as a whole or within one or more regions of the device. The device is not limited to any particular profile and encompasses any profile.

In an embodiment, the surfaces of the device may be modified to improve the mechanical integrity of the device and/or improve patient comfort. For example, in one embodiment, the outer surface of the device may be cast, molded, shaped and/or polished to achieve a smooth surface. Without wishing to be bound by theory, a smooth surface may lower the coefficient of friction for the device. Because the outer surface generally contacts the under surface of the eyelid, providing a smooth surface may improve comfort for the patient and may reduce dislodgment of the device by allowing the eyelid to slide smoothly over the surface of the device. In still other embodiments, a surface or a portion of a surface of the device may be shaped, molded, cast and/or machined to be rough or rougher than another portion of the device. For example, a rough surface on the underside of the device which may contact the conjunctiva or exposed sclera of the eye may provide mechanical keying or improved coherence to the surface of the eye and may impart improved stability during use. A rough surface oil an upper surface or a portion of an upper surface may improve ease of handling of the device.

Optionally, in an embodiment, the device may include one or more openings for a surgical instrument. A surgical instrument may include, but is not limited to, an injection needle or a trocar. In an embodiment, the opening may allow an injection to occur while the device is located on the eye. The openings may ensure that no portion of the device is injected into the eye by an inserted surgical instrument. In an embodiment, the openings may only be large enough to allow the injection needle, or other surgical instrument, to enter. In an embodiment, an injection needle may range in size from, for example, a 20-gauge needle to a 31-gauge needle. In an embodiment, the device may have a single opening. The device may be rotated to allow for more than one injection. Alternatively, the device may have an opening at each site where an injection can be given. In an embodiment, the device may have four openings.

Optionally, in an embodiment, the device may include an alignment feature. The alignment feature may include, but is not limited to, a line, a hole, a notch, a coloring, a dent and/or an impression. The alignment feature may be an axis line on the device. The alignment feature may be used to align the device with an injection site. The alignment feature may be used to center the device on the eye. In an embodiment, the alignment feature may be used in positioning the device based on the base radius of curvature.

A method is also provided for preventing or reducing an incidence of endophthalmitis following micro-incision surgery by providing an active agent, such as, an antibiotic to at least a portion of the effected area. This method may also reduce the incidence of one or more additional complications associated with, for example, micro-incision vitrectomy. While the is designed for delivery of an active agent to the site of surgery or microsurgery, it is contemplated that the device may be utilized in any method requiring delivery of an agent to an eye. For example, the device may be used to deliver antibiotic, anti-viral or antifungal agents to a diseased eye. Moreover, different devices may be utilized based on the use and the type or form of the active agent. For example, the thickness or composition of the material for a device may be selected to accommodate active agents having differing chemistries of forms and/or to accommodate a specific dosage of the active agent.

The method may include placing the ophthalmic medication/antibiotic delivery device on the surface of an eye of a patient following vitrectomy surgery, where the collagen shield is positioned evenly around the cornea to cover the surgical incisions of vitrectomy posterior to the corneal edge. In a complicated ophthalmic surgical case, where a combination of a vitrectomy surgery and a surgery involving incisions to the cornea, such as in cataract surgery, are preformed, the method would preferably use a large collagen shield disc that covers both the surgical incisions on the conjunctiva as well as the incisions on the cornea.

The method may place the device on the surface of the eye. The device may include a collagen shield having an annular configuration with an inner periphery that approximates the circumference of the cornea. In an embodiment, the device may be placed on the surface of the eye in such a manner that substantially maximum contact (maximum pressure) is on the perilimbal conjunctiva (conjunctiva 360 degrees around the cornea), leaving the cornea substantially exposed or uncovered by the device. However, as discussed above, other embodiments may cover a portion of the cornea of the eye in addition to covering the sites of micro-surgical incisions to the sclera.

In an embodiment, the annular device may be placed on the surface of an eye such that the device surrounds, but does not cover, the cornea and covers a site or an incision in the sclera without extending past the edge of the bulbar conjunctiva. The device may include a biocompatible material and may substantially conform to a curvature of the eye. In an embodiment, a needle may be received through one or more openings of the device.

In an embodiment, an ophthalmic antibiotic solution or suspension may be applied to the collagen shield, such as by presoaking, prior to placement of the ophthalmic antibiotic delivery device on the eye. The soak time and the nature and duration of the soak may be determined by the application at hand, the patient's condition and the treating physician's assessment of the patient's needs. In an embodiment, the device may be soaked to absorb ophthalmic medication. In an embodiment, the ophthalmic medication may be released over a period of time to at least an incision site when the device is placed on the surface of the eye. In an embodiment, an anesthetic may be applied to the device.

In an embodiment, the ophthalmic medication/antibiotic delivery device may provide a slow-release of therapeutic doses of antibiotics and wound tamponade to open sclerotomies following minimally-invasive posterior segment eye surgery. This may provide therapeutic and detectable levels of antibiotic in the posterior segment and in the anterior segment of the eye. In an embodiment, the device may avoid irritation and trauma to the cornea.

Additionally, the device and method for use may provide for physical healing and treatment of wounds resulting from ophthalmic procedures. For example, the device may be used to cause and improve the eye's recovery from surgical wounds, including wounds from surgery.

Having now described various embodiments of the device, it will be appreciated that a wide range of equivalent parameters, concentrations, geometries, sizes, and conditions are possible to construct and use without departing from the spirit and scope of the invention and without undue experimentation.

While the device has been described in connection with specific embodiments thereof, it will be understood that it is capable of further modifications. This application is intended to cover any variations, uses, or adaptations of the device following, in general, the principle discussed and including such departures from the present disclosure as come within known or customary practice within the art to which the device pertains and as may be applied to the essential features hereinbefore set forth as follows in the scope of the appended claims.

Reference to known method steps, conventional methods steps, known methods or conventional methods is not in any way an admission that any aspect, description or embodiment of the present invention is disclosed, taught or suggested in the relevant art.

What is claimed is:

1. A method of administering ophthalmic medication to a site of one or more incisions in a sclera of an eye comprising applying an annular device to the site, wherein the device comprises a biocompatible material, an inner edge and an outer edge, wherein the inner edge surrounds a cornea of the eye without covering the cornea, wherein the outer edge covers the site of the one or more incisions without extending past an edge of a bulbar conjunctiva of the eye, and wherein the device releases the ophthalmic medication to at least the site of the one or more incisions.

2. The method of claim 1, further comprising:
soaking the device to absorb ophthalmic medication.

3. The method of claim 1, further comprising:
applying an anesthetic to the device.

4. The method of claim 1, wherein the biocompatible material comprises collagen.

5. The method of claim 4, wherein the collagen comprises crosslinked collagen and partially hydrolyzed collagen.

6. The method of claim 1, wherein the biocompatible material comprises high purity type I collagen.

7. The method of claim 1, wherein the inner edge defines a substantially circular shaped central opening having a diameter between about 10 millimeters and about 14 millimeters.

8. The method of claim 1, wherein the outer edge forms a substantially circular shape having a diameter between about 14 millimeters and about 26 millimeters.

9. The method of claim 1, wherein a distance between the inner edge and the outer edge is between about 2 millimeters and about 8 millimeters.

10. An annular device comprising a biocompatible material, wherein the device is configured to:
substantially conform to a curvature of an eye; and
be placed on a surface of the eye such that:
the device surrounds a cornea of the eye without covering the cornea, and
the device covers a site of one or more incisions in a sclera of the eye without extending past an edge of a bulbar conjunctiva of the eye.

11. The device of claim 10, wherein the biocompatible material comprises collagen.

12. The device of claim 11, wherein the collagen comprises crosslinked collagen and partially hydrolyzed collagen.

13. The device of claim 10, wherein the biocompatible material comprises high purity type I collagen.

14. The device of claim 10, wherein the biocompatible material comprises at least 95% type I collagen.

15. The device of claim 10, wherein the biocompatible material comprises at least 98% type I collagen.

16. The device of claim 10, wherein the device comprises an inner edge, wherein the inner edge defines a substantially circular shaped central opening having a diameter between about 10 millimeters and about 14 millimeters.

17. The device of claim 10, wherein the device comprises an inner edge, wherein the inner edge defines a substantially circular shaped central opening having a diameter greater than about 14 millimeters.

18. The device of claim 10, wherein the device comprises an outer edge, wherein the outer edge forms a substantially circular shape having a diameter between about 14 millimeters and about 26 millimeters.

19. The device of claim 10, wherein the device comprises an outer edge, wherein the outer edge forms a substantially circular shape having a diameter between about 24 millimeters and about 25 millimeters.

20. The device of claim 10, wherein the device comprises an inner edge and an outer edge, wherein a distance between the inner edge and the outer edge is about 6 millimeters.

21. The device of claim 10, wherein the device comprises an inner edge and an outer edge, wherein a distance between the inner edge and the outer edge is about 10 millimeters.

22. The device of claim 10, wherein the device comprises a caruncular cutout.

23. The device of claim 10, wherein the device comprises an absorbed amount of ophthalmic medication.

24. The device of claim 23, wherein the device is further configured to release the ophthalmic medication over a period of time to at least the site of the one or more incisions when the device is placed on the surface of the eye.

25. The device of claim 10, wherein the device comprises an antimicrobial agent.

26. The device of claim 10, wherein the device comprises an anesthetic.

27. The device of claim 10, wherein the device is microbicidal.

28. The device of claim 10, wherein the device is microbistatic.

29. The device of claim 10, wherein the device is further configured to be transparent and translucent such that the site of the one or more incisions in the sclera of the eye may be viewed through the device.

30. The device of claim 10, wherein the device is further configured to dissolve within an amount of time, wherein the amount of time is between about 12 hours and about 72 hours.

31. The device of claim 10, wherein the device conforms to contours of the sclera of the eye.

32. The device of claim 10, wherein the device comprises a sclera base radius of curvature that conforms to contours of the sclera of the eye.

33. The device of claim 10, wherein the device comprises a sclera base radius of curvature between about 9 millimeters and about 13 millimeters.

34. The device of claim 10, wherein the device further comprises one or more openings, wherein each opening is configured to receive a surgical instrument.

35. The device of claim 10, wherein the device comprises an inner edge and an outer edge, and wherein the device further comprises an acute tapered angle between about 0 degrees and about 90 degrees from the outer edge to the inner edge along a central axial length corresponding to a line of sight of the eye.

36. The device of claim 10, wherein the device comprises an alignment feature.

37. An annular device comprising a biocompatible material and an ophthalmic medication, the device having an inner edge and an outer edge, wherein the device is configured to:
substantially conform to a curvature of an eye;
be placed on a surface of the eye such that:
the inner edge surrounds a cornea of the eye without covering the cornea, and
the outer edge covers a site of one or more incisions in a sclera of the eye without extending past an edge of the a bulbar conjunctiva of the eye; and
release the ophthalmic medication over a period of time to at least the site of the one or more incisions when the device is placed on the surface of the eye.

38. The device of claim 37, wherein the inner edge defines a substantially circular shaped central opening having a diameter between about 10 millimeters and about 14 millimeters.

39. The device of claim 37, wherein the outer edge forms a substantially circular shape having a diameter between about 14 millimeters and about 26 millimeters.

40. The device of claim 37, wherein a distance between the inner edge and the outer edge is between about 2 millimeters and about 8 millimeters.

41. An annular device comprising a biocompatible material and an absorbed amount of an ophthalmic medication, the device having:
an inner edge, wherein the inner edge has a diameter of at least about 10 millimeters; and
an outer edge, wherein the outer edge has a diameter of no more than about 26 millimeters;
wherein the device is configured to:
substantially conform to a curvature of an eye,
be placed on a surface of the eye such that:
the inner edge surrounds a cornea of the eye without covering the cornea, and
the outer edge covers a site of one or more incisions in a sclera of the eye without extending past an edge of a bulbar conjunctiva of the eye, and
release the ophthalmic medication over a period of time to at least the site of the one or more incisions when the device is placed on the surface of the eye.
